(19) United States
(12) Patent Application Publication
(10) Pub. No.: US 2026/0033951 A1
(43) Pub. Date: Feb. 5, 2026

(54) HYBRID INLAY/ONLAY ARTICULAR SURFACE IMPLANTS AND METHODS

(71) Applicant: Arthrosurface Incorporated, West Bridgewater, MA (US)

(72) Inventors: Steven W. Ek, Bolton, MA (US); Anthony Miniaci, Bentleyville, OH (US); Timothy H. Brightman, Franklin, MA (US)

(21) Appl. No.: 19/354,810

(22) Filed: Oct. 9, 2025

Related U.S. Application Data
(62) Division of application No. 17/425,400, now abandoned.
(60) Provisional application No. 62/796,350, filed on Jan. 24, 2019.

(51) Int. Cl. A61F 2/30; A61F 2/40

(52) U.S. Cl. CPC A61F 2/30756; A61F 2/30749; A61F 2/4081; A61F 2002/30326; A61F 2002/30332

(57) ABSTRACT

A hybrid inlay/onlay (HIO) implant system includes an implant and an anchor. The implant comprises an inlay component and an onlay component. The inlay component is configured to be at least partially received in an excision site and includes a bone facing surface configured to abut against the bone within the excision site. The onlay component comprises an uppermost surface and a stepped surface. The uppermost surface includes an articulating surface configured to articulate against an articulating surface associated with a second bone of the joint. The stepped surface is configured to abut against at least some of a remaining portion of the patient's native articular surface proximate the excision site and/or a peripheral step excision site formed proximate the excision site. The anchor is configured to extend away from the bone facing surface of the inlay segment and to secure the HIO implant system to the first bone.

### FIELD

The present disclosure is related to devices and methods for the repair of defects that occur in articular cartilage on the surface of bones, and particularly to hybrid inlay/onlay articular surface implants and methods.

### BACKGROUND

Articular cartilage, found at the ends of articulating bones in the body, is typically composed of hyaline cartilage, which has many unique properties that allow it to function effectively as a smooth and lubricious load-bearing surface. When injured, however, hyaline cartilage cells are not typically replaced by new hyaline cartilage cells. Healing is dependent upon the occurrence of bleeding from the underlying bone and formation of scar or reparative cartilage called fibrocartilage. While similar, fibrocartilage does not possess the same unique aspect of native hyaline cartilage and tends to be less durable.

In some cases, it may be necessary or desirable to repair the damaged articular cartilage using one or more implants. While implants may be successfully used, the implant should be designed to maximize the patient's comfort, minimize damage to surrounding areas, minimize potential further injury, maximize the functional life of the implant, and be easy to install.

### BRIEF DESCRIPTION OF THE DRAWINGS

Features and advantages of the present invention are set forth by description of embodiments consistent with the present invention, which description should be considered in conjunction with the accompanying drawings wherein:

FIG. 1 generally illustrates one example of a hybrid inlay/onlay (HIO) implant system consistent with the present disclosure implanted in the glenoid, with the glenoid in cross-section along the medial-lateral (ML) plane;

FIG. 2 generally illustrates the HIO implant system implanted in the glenoid with the glenoid in cross-section along the superior-inferior (SI) plane;

FIG. 3 is a close-up of the region III in FIG. 1 in the ML plane;

FIG. 4 is a close-up of the region II in FIG. 2 in the SI plane;

FIG. 5 generally illustrates the HIO implant system of FIGS. 1-4 in an exploded or unassembled state;

FIG. 6 generally illustrates the HIO implant system of FIG. 5 in an assembled state;

FIG. 7 is a cross-sectional view of the implant taken along the ML plane;

FIG. 8 is a cross-sectional view of the implant taken along the DI plane;

FIG. 9 generally illustrates a cross-sectional view of the excision site along the ML plane;

FIG. 10 generally illustrates a cross-sectional view of the excision site along the SI plane;

FIG. 11 is a perspective bottom view of one example of an implant consistent with the present disclosure;

FIG. 12 is a perspective top view of the implant of FIG. 11 consistent with the present disclosure;

FIG. 13 is a perspective top view of one example of a post consistent with the present disclosure; and

FIG. 14 generally illustrates a cross-sectional view of the excision site along the SI plane including a stepped region.

### DETAILED DESCRIPTION

By way of an overview, one aspect of the present disclosure features systems and methods for repairing all or a portion of an articular surface associated with a bone of a joint. The joint may include any joint such as, but not limited to, a shoulder joint, a talocrural joint, a knee joint, a toe joint, a finger joint, or the like. As described herein, the systems and methods include an implant system for a glenoid. The implant system may a hybrid inlay/onlay (HIO) implant system for replacing at least a portion of a patient's native articular surface on a first bone in a joint. The HIO implant system may include an implant and an anchor. The implant may comprise an inlay component and an onlay component. The inlay component may be configured to be at least partially received in an excision site formed in the first bone beneath a removed portion of a patient's native articular surface. The inlay segment may include a bone facing surface configured to abut against the bone within the excision site. The onlay component may comprise an uppermost surface and a stepped surface (e.g., but not limited to, an articular surface facing surface). The uppermost surface includes an articulating surface configured to articulate against an articulating surface associated with a second bone of the joint. The stepped surface is configured to abut against at least some of a remaining portion of the patient's native articular surface proximate to the excision site and/or a peripheral step excision site formed proximate to the excision site. The anchor is configured to extend away from the bone facing surface of the inlay segment and to secure the HIO implant system to the first bone.

The present disclosure may therefore feature an prothesis (implant system) which is particularly suited for repairing a patient's glenoid. The implant system may provide the benefits of increased coverage, while minimizing the amount of bone removed, reducing lateralization, and increasing the stability of the implant system (thereby reducing the likelihood of implant dislocation)

In particular, one or more aspects of the present disclosure may feature a glenoid HIO implant system. It should be appreciated that the HIO implant system described herein is not limited to the glenoid unless specifically claimed as such, and a HIO implant system consistent with one or more embodiments of the present disclosure may be installed in any joint which translates on another articular surface.

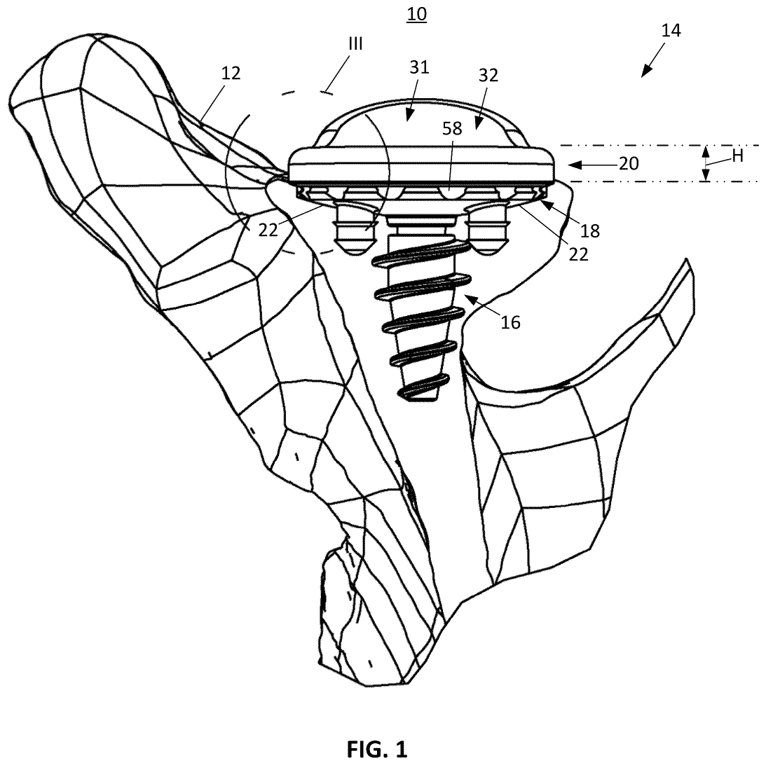

Turning to FIGS. 1-4, one example of a hybrid inlay/onlay implant system 10 (also generally referred to as HIO implant system 10) is generally illustrated implanted in a patient's glenoid 12. While the HIO implant system 10 may be shown and described in the context of a glenoid HIO implant system, it should be appreciated that one or more HIO implant systems 10 consistent with the present disclosure may be installed in any other joint including, but not limited to, any ball-and-socket joints (e.g., a hip joint, a shoulder joint, or the like), any hinge joints (e.g., an elbow joint, a knee joint, or the like), any condyloid joints (e.g., a wrist joint), or the like.

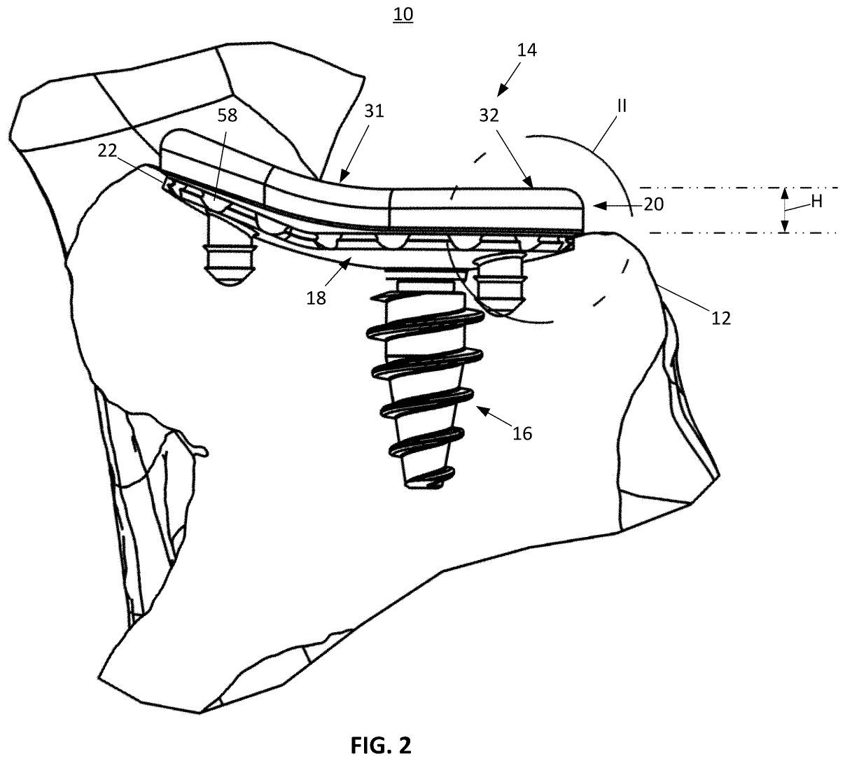

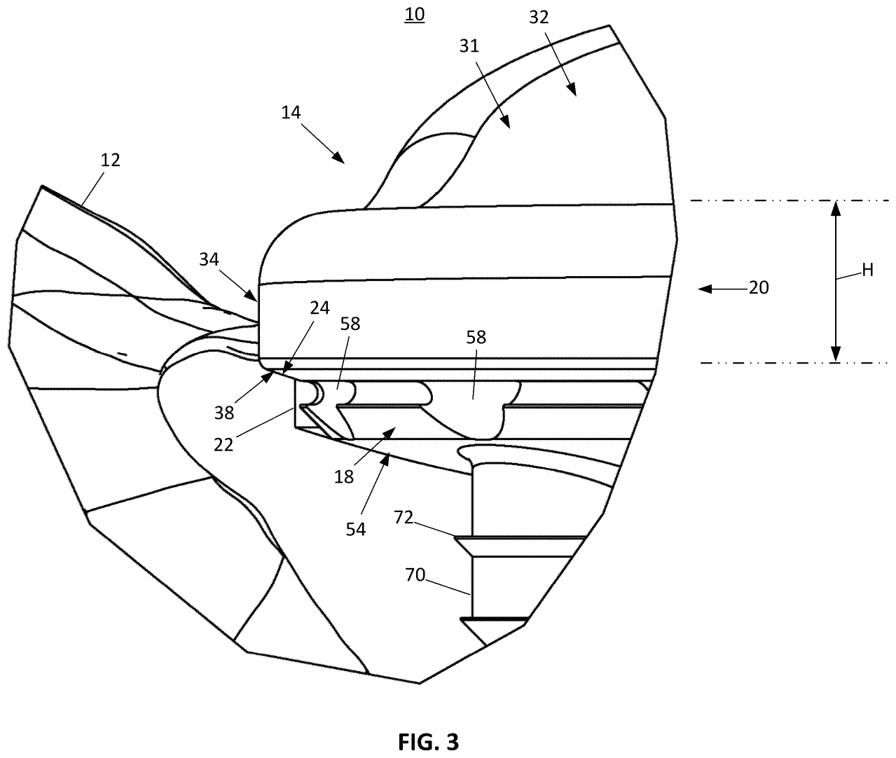

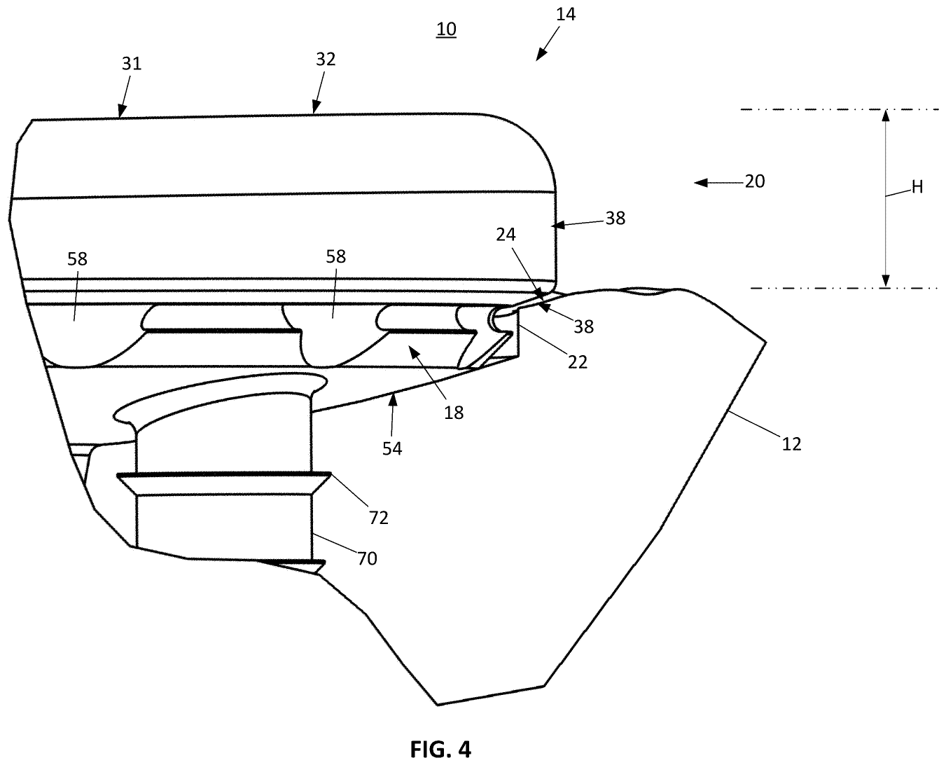

In particular, FIG. 1 generally illustrates the HIO implant system 10 implanted in the glenoid 12 with the glenoid 12 in cross-section along the medial-lateral (ML) plane, FIG. 2 generally illustrates the HIO implant system 10 implanted in the glenoid 12 with the glenoid 12 in cross-section along the superior-inferior (SI) plane, FIG. 3 is a close-up of the region III in FIG. 1 in the ML plane, and FIG. 4 is a close-up of the region II in FIG. 2 in the SI plane. As explained herein, the HIO implant system 10 may include an implant 14 and one or more anchors 16 configured to secure the implant 14 to the bone 12 (e.g., glenoid). The implant 14 may include an inlay component 18 and an onlay component 20. The inlay component 18 and onlay component 20 may be formed as a single piece (e.g., monolithic) or two or more pieces coupled (e.g., either permanently or removably) together. The onlay component 20 may have a length and/or width that is larger than the length and/or width, respectively, of the inlay component 18. For example, in at least one example, the HIO implant system 10 may be configured such that when secured to an excision site 22 formed in the glenoid 12, the inlay component 18 is at least partially received in the excision site 22, while the onlay component 20 extends over at least a portion of the remaining native articular surface 24 of the glenoid 12.

Because the inlay component 18 is configured to be at least partially received in an excision site 22 formed in the glenoid 12, a HIO implant system 10 consistent with at least one example of the present disclosure may be secured to the glenoid 14 more robustly compared to known onlay glenoid implants which are disposed substantially entirely on the native articular surface of the glenoid. As a result, the HIO implant system 10 is less likely to shift after implantation, thereby reducing the potential for further damage to the patient's joint and/or reducing the need for further corrective surgery. In addition, because the inlay component 18 is configured to be at least partially received in the excision site 22, a HIO implant system 10 consistent with at least one example of the present disclosure may reduce the protruding height H that the implant 14 extends above the patient's native articular surface 24 compared to known onlay glenoid implants. Reducing the protruding height H of the implant 14 has the benefit of reducing the amount of lateralization associated with the known onlay glenoid implants.

Moreover, because the onlay component 20 has a larger length and/or width than the length and/or width, respectively, of the inlay component 18, a HIO implant system 10 consistent with at least one example of the present disclosure may provide increased coverage compared to known inlay glenoid components. In particular, the onlay component 20 may allow the HIO implant system 10 to replace a larger (e.g., all) of the patient's native articular surface 24 with a minimal amount of the glenoid being removed. As such, the risk of weakening the glenoid due to extensive reaming may be minimized, while also increasing the coverage of the HIO implant system 10 compared to known glenoid implants.

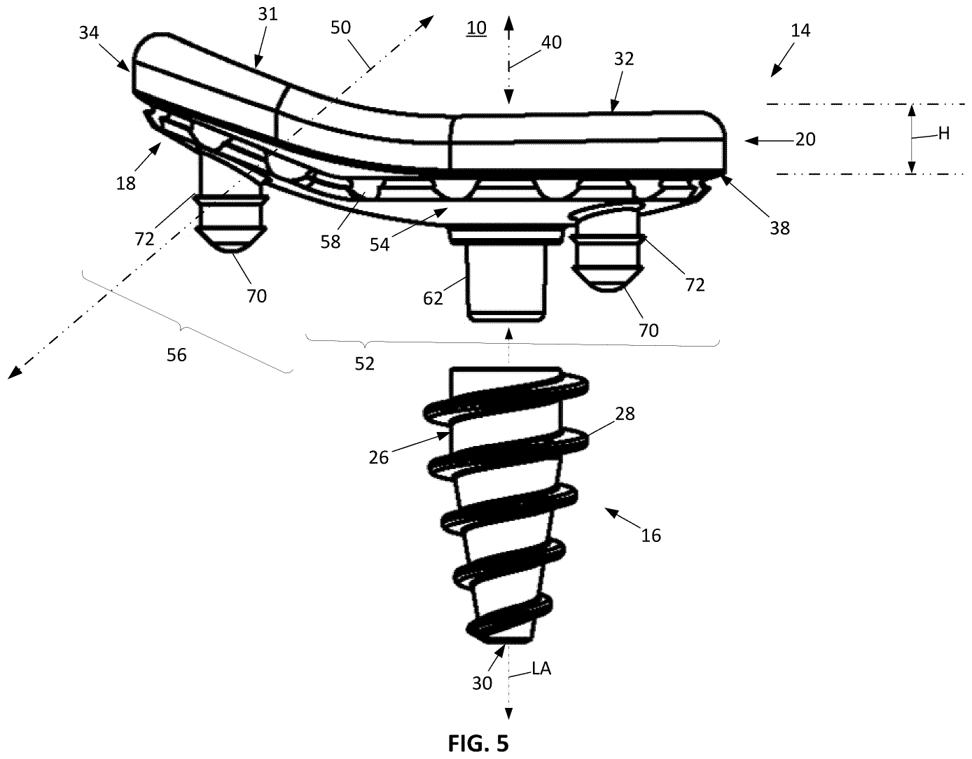

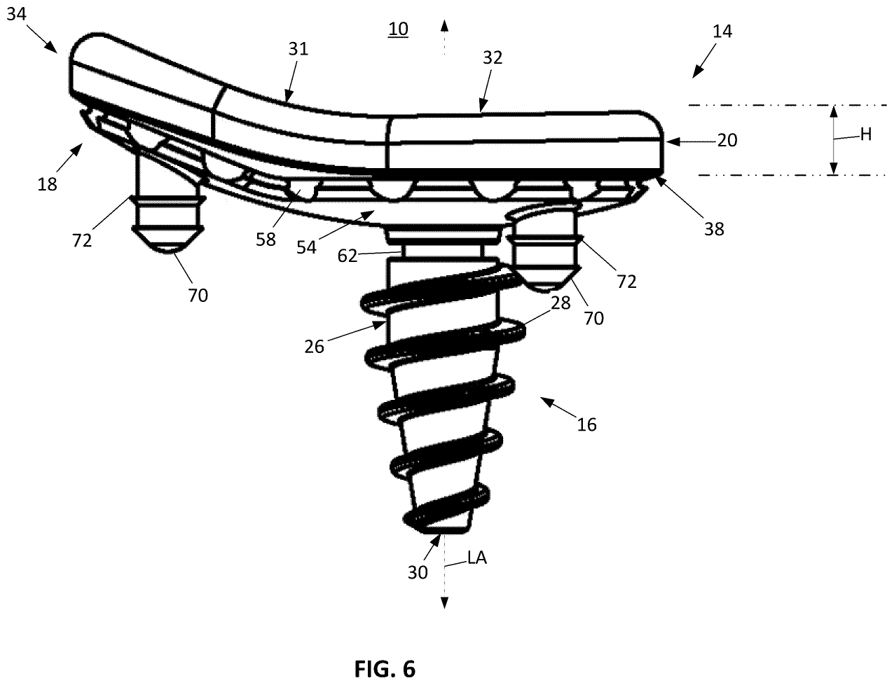

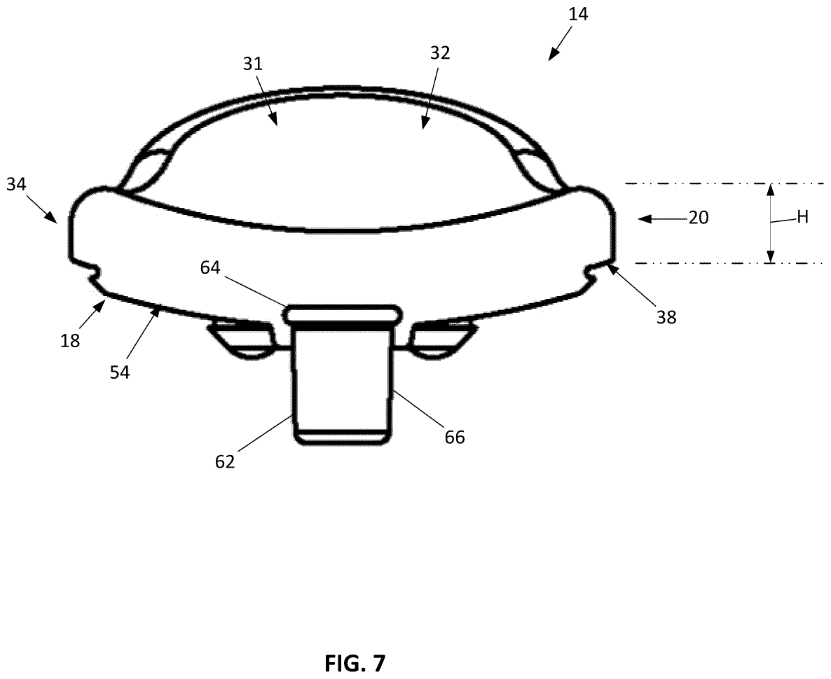

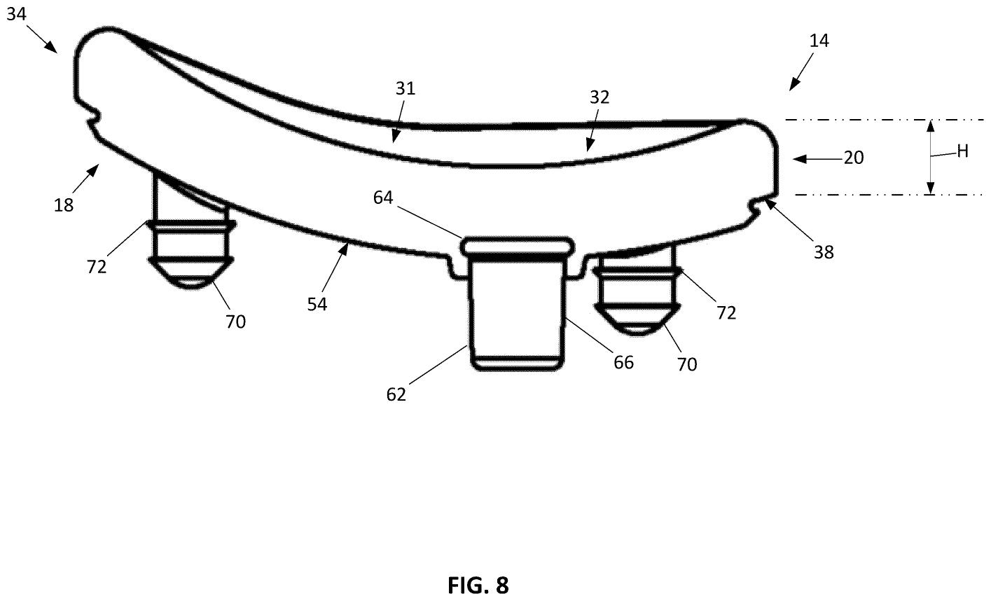

Turning to now FIGS. 5-8, one example of the HIO implant system 10 of FIGS. 1-4 is shown without the bone 12. In particular, FIG. 5 generally illustrates the HIO implant system 10 in an exploded or unassembled state, FIG. 6 generally illustrates the HIO implant system 10 in an assembled state, FIG. 7 is a cross-sectional view of the implant 14 taken along the ML plane, and FIG. 8 is a cross-sectional view of the implant 14 taken along the DI plane.

As noted above, the HIO implant system 10 includes an implant 14 and one or more anchors 16 configured to secure the implant 14 to the bone 12. The anchor 16 may be removably coupled to the implant 14. For example, the anchor 16 may be removably coupled to the implant 14 using any connection such as, but not limited to, a tapered connection (e.g., a Morse taper) or the like. Alternatively, the anchor 16 may be non-removably coupled to and/or part of the implant 14.

The exterior surface 26 of the anchor 16 may include one or more threads, ribs, protrusions, undercuts, recesses, or the like 28 configured to engage and generally retain the anchor 16 to the bone 12. In at least one example, the anchor 16 may be configured to be secured to bone 12 within the excision site 22. One benefit of the threads 28 is that the position of the anchor 16 within the excision site 22 (and the resulting position of the implant 14) may be adjusted such that the onlay component 20 abuts against the patient's remaining native articular surface 24. Optionally, the anchor 16 may include a cannulated passageway 30 extending end-to-end along its longitudinal axis LA.

The implant 14 may include an inlay component 18 configured to be at least partially received in the excision site 22 and an onlay component 20 configured to extend over at least a portion of the remaining native articular surface 24 of the glenoid 12. The onlay component 20 may include an uppermost, exposed surface 31, at least a portion of which includes an articulating surface 32 configured to articulate against an articulating surface associated with a second bone in the joint (such as, but not limited to, an articulating surface associated with the humerus). The bounds of the articulating surface 32 may correspond to the amount of the patient's native articular surface 24 that is to be replaced. In at least one example, the articulating surface 32 may replace all of the patient's native articular surface 24 on the bone 12.

The articulating surface associated with the second bone in the joint may include the patient's native articular surface and/or an articulating surface of an implant secured to the second bone. The articulating surface 32 of the implant 14 may have a contour/profile configured to articulate against the articulating surface associated with the second bone in the joint. For example, the articulating surface 32 of the implant 14 may have a contour/profile which substantially corresponds to the portion of the patient's native articular surface being replaced. As used herein, “substantially corresponds” means that the contour/profile of the articulating surface 32 is within 15% of the contour/profile of the patient's native articular surface being replaced. Alternatively, the articulating surface 32 may have a contour/profile based on the contour/profile of the articulating surface associated with the second bone in the joint. To this end, the contour/profile of the articulating surface 32 may not correspond to the contour/profile of the patient's native articular surface being replaced.

Whereas known inlay implants include an articulating surface which is flush with the patient's native articular surface proximate to the excision site, the articulating surface 32 of the onlay component 20 is raised relative to the patient's native articular surface. To this end, the onlay component 20 may include a sidewall 34 extending around its periphery 36. The sidewall 34 may extend from the uppermost exposed surface 31 of the onlay component 20 to a stepped surface 38. The sidewall 34 may have a height corresponding to a height or thickness of the onlay component 20 between the uppermost exposed surface 31 and the stepped surface 38. In at least one example, the height of the sidewall 34 may correspond to the height H that the implant 14 extends above the patient's native articular surface 24. The thickness of the onlay component 20 may also be selected to minimize the amount of lateralization when the implant system 10 is implanted in the patient while also ensuring that the implant 14 does not deform and/or break during normal use when implanted in the patient. By way of a non-limiting example, the height H may be between 3-6 mm, e.g., between 4-5 mm, such as 4.5 mm.

The articulating surface 32 of the onlay component 20 may therefore extend over at least a portion of the patient's native articular surface proximate to the excision site 22. As a result, the articulating surface 32 of the implant 14 may extend across a larger area than the inlay component 18 and/or the excision site 22 formed in the glenoid 12. For example, the width Wo and/or length Lo of the onlay component 20 may be larger than the width Wi and/or length Li, respectively, of the inlay component 18. The width Wo and/or length Lo of the onlay component 20 may also be larger than the width We and/or length Le, respectively, of the excision site 22. As used herein, the widths Wo, Wi are intended to refer to the maximum linear distances of the onlay component 18 and the inlay component 20, respectively, between two opposing sides of a cross-section of the implant in the ML plane while the lengths Lo, Li are intended to refer to the maximum linear distances of the onlay component 18 and the inlay component 20, respectively, between two opposing sides of a cross-section of the implant in the SI plane. Similarly, the width We is intended to refer to the maximum linear distance of the excision site 22 between two opposing sides of a cross-section of the excision site 22 in the ML plane while the length Le is intended to refer to the maximum linear distance of the excision site 22 between two opposing sides of a cross-section of the excision site 22 in the SI plane. The width Wo and length Lo of the onlay component 20 may be equal to the width Wi and length Li of the inlay component 18 plus the length of the stepped surface 38 in the ML and SI planes, respectively. For illustrative purposes, the onlay component 20 may have a width Wo that is 2-15 mm larger than the width W1 of the inlay component 18 and/or may have a length Lo that is 2-15 mm larger than the length Li of the inlay component 18. For example, the width Wo may be 4-10 mm larger than the width Wi and/or the length Lo may be 4-10 mm larger than the length Li.

According to one example, the stepped surface 38 may extend over a portion of the bone adjacent to the patient's articular surface 24. The stepped surface 38 may form a shoulder and/or step extending along a transition between the larger cross-sectional onlay component 20 and the inlay component 18. The stepped surface 38 may have a contour/profile configured to allow the articular surface facing surface 38 to substantially coplanar with the portion of the patient's articular surface 24 and/or bone 12 proximate to the excision site 22. In at least one example, the stepped surface 38 may include an articular surface facing surface configured to abut against a portion of the patient's articular surface 24 proximate to the excision site 22. The articular surface facing surface 38 may have a contour/profile substantially corresponding to the contour/profile of the portion of the patient's articular surface 24 proximate to the excision site 22.

In at least one example, the stepped surface 38 may form a planar surface. The stepped surface 38 may include a generally tapered configuration. For example, the generally tapered stepped surface 38 may taper inwardly as one moves closer to the inlay component 18 and/or may taper outwardly as one moves closer to the inlay component 18. The taper of the stepped surface 38 may be linear and/or nonlinear. In at least one example, the tapered stepped surface 38 may be formed by one or more overlapping generally frustoconical shapes. Alternatively (or in addition), the stepped surface 38 may include a planar cross-section.

The inlay component 18 may be configured to be at least partially received in one or more excision sites 22 formed the bone 12. As noted herein, the inlay component 18 may function (along with the anchor 16) to secure the implant 14 to the bone 12. In particular, the inlay component 18 may have a size and shape generally inversely corresponding to the size and shape of the excision site 22. The size and shape of the inlay component 18 is configured to interact with the excision site 22 to generally prevent side-to-side movement of the implant 14 within the excision site 22.

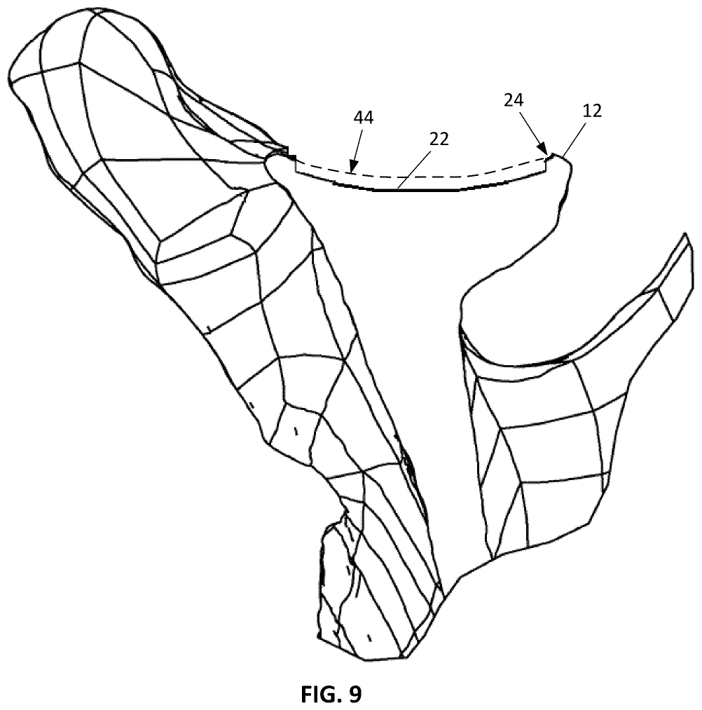

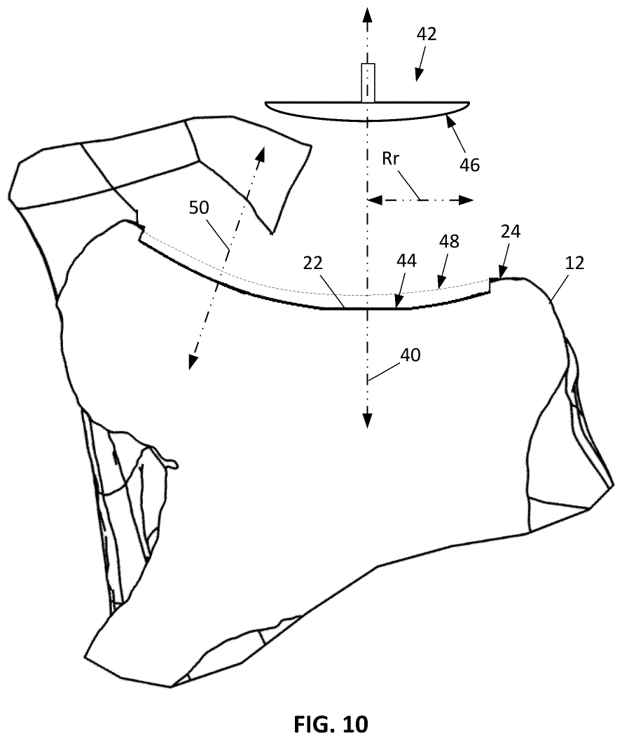

With further reference to FIGS. 9 and 10, cross-sectional views an exemplary example of an excision site 22 are generally illustrated. In particular, FIG. 9 generally illustrates a cross-sectional view of the excision site 22 along the ML plane and FIG. 10 generally illustrates a cross-sectional view of the excision site 22 along the SI plane. The excision site 22 may be formed in any manner known to those skilled in the art. For example, at least a portion of the excision site 22 may be formed by advancing a first reamer 42 along a first or cutting axis 40 (FIG. 10). The reamer 42 may rotate about the first axis 40 to form a first portion of the excision site 22 having a generally circular cross-section. The base 44 of the excision site 22 may have a contour corresponding to the contour/profile of the cutting surface 46 of the reamer 42 rotated/revolved about the first axis 40. The first axis 40 may be perpendicular to the patient's original articular surface 48 which is being removed (which is generally illustrated by the dotted line) and/or at an acute angle. Optionally, one or more additional portions of the excision site 22 may be formed by advancing a reamer 42 (which may have the same or different radius and/or profile as the first reamer 42) along one or more additional axes 50. The additional axes 50 may be parallel to the first axis 40 and/or at an acute angle relative to the first axis 40. The additional axes 50 may be spaced apart from the first axis 40 such that the additional portions of the excision site 22 formed when the reamer 42 is advanced along the additional axes 50 partially overlap with an adjacent portion of the excision site 22.

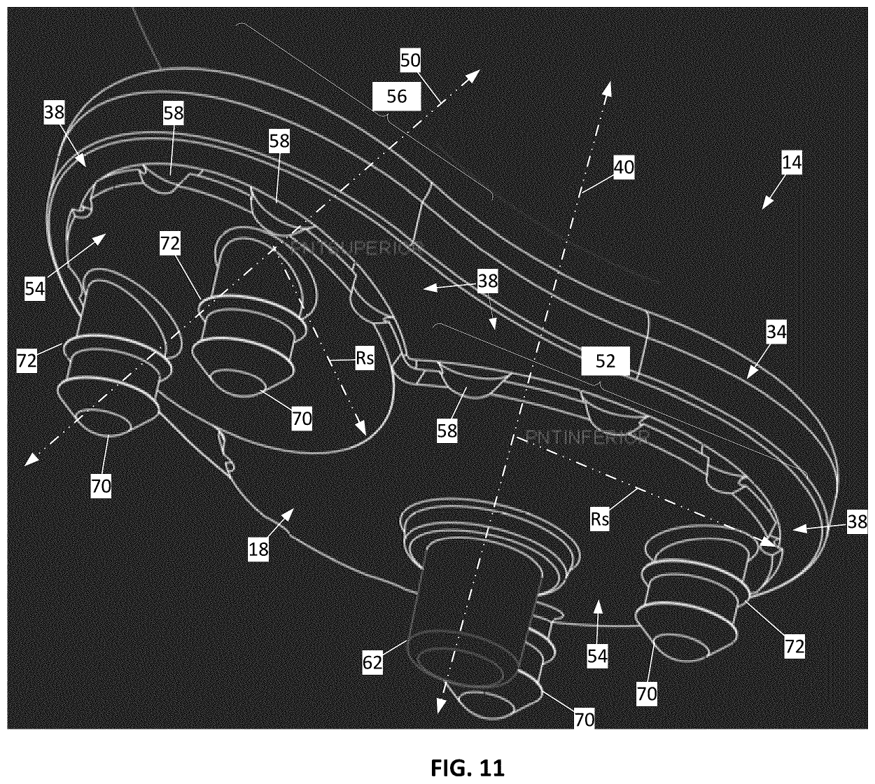

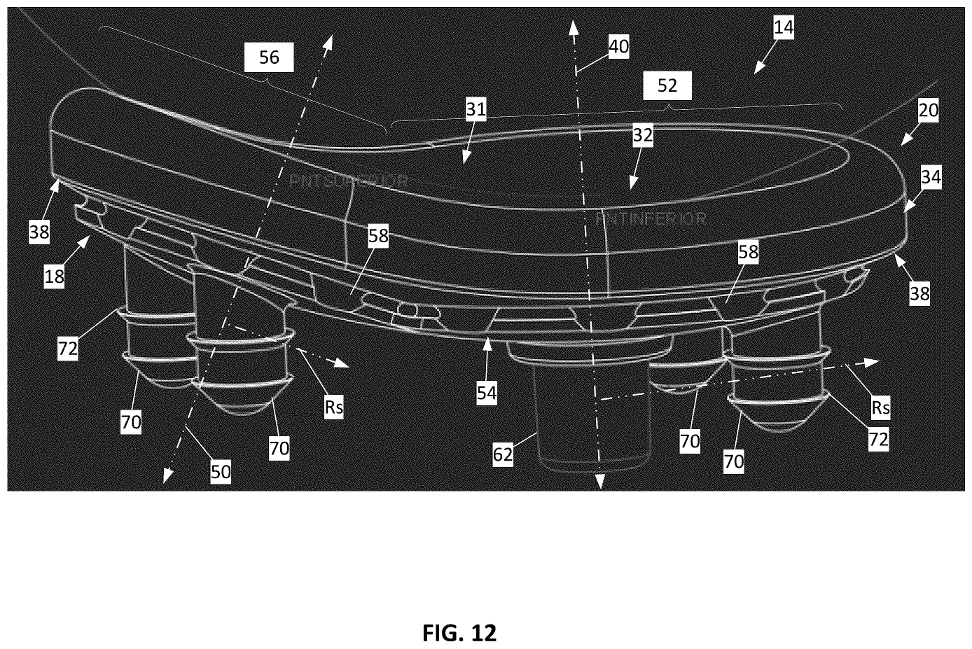

With reference now to FIGS. 11 and 12, a perspective bottom view and a perspective top view of the implant 14 (including additional pegs as discussed herein) is generally illustrated. In particular, it can be seen that the inlay component 18 includes a first inlay segment 52 which is rotated/revolved about the first axis 40. At least a portion of the first inlay segment 52 may include a radius Rs which corresponds to the radius Rr (FIG. 10) of the reamer 42. As discussed herein, the contour/profile of the bone facing surface 54 of the first inlay segment 52 may generally correspond to the contour/profile of the cutting surface 46 of the reamer 42 rotated/revolved about the first axis 40. In at least one example, the first inlay segment 52 may include a generally concaved and/or generally hemi-spherical configuration.

Optionally, the inlay component 18 may include one or more additional (e.g., but not limited to, second) inlay segment(s) 56 which are rotated/revolved about the additional axes 50. At least a portion of the additional inlay segment(s) 56 may include a radius Rs which corresponds to the radius Rr of the reamer 42 that is rotated/revolved about the additional axes 50. The contour/profile of the bone facing surface 54 of the additional inlay segment(s) 56 may generally correspond to the contour/profile of the cutting surface 46 of the reamer 42 that is rotated/revolved about the additional axes 40. In at least one example, the additional inlay segment 56 may include a generally concaved and/or generally hemi-spherical configuration. As can be seen, the first inlay segment 52 and the additional inlay segment 56 partially overlap in the same manner as the first and additional portions of the excision site 22. In at least one example, the first inlay segment 52 and/or additional inlay segment 56 may include a generally concaved and/or generally hemi-spherical configuration. For example, the

At least a portion of the bone facing surface 54 of the inlay component 18 may include one or more recesses, cavities, and/or undercuts 58. The undercuts 58 may form regions for bone cement to adhere to both the inlay component 18 and the bone 12 to further aid in securing the implant 14 to the bone 12. In at least one example, the undercuts 58 may extend along all or a portion of a periphery 60 of the inlay component 18 adjacent to the articular surface facing surface 38.

The implant 14 may optionally include one or more stems and/or posts 62. The post 62 may extend away from the bone facing surface 54 of the inlay component 18. In the illustrated example, the post 62 is shown extending away from the bone facing surface 54 of the first segment 52 of the inlay component 18; however, it should be appreciated that one or more posts 62 may alternatively or additionally extend from the bone facing surface 54 of the additional segment(s) 56 of the inlay component 18. In at least one example, a longitudinal axis of the post 62 may be colinear with the first and/or additional axes 40, 50, though this is not a limitation of the present disclosure unless specifically claimed as such.

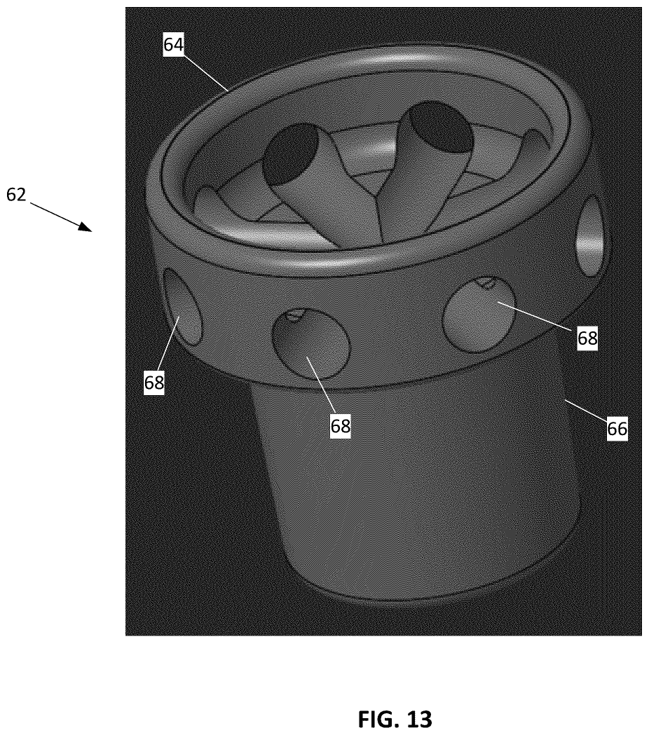

The post 62 may be formed from a metal such as, but not limited to, chromium alloys, titanium alloys, stainless steel alloys, or the like. The post 62 may be overmolded within the implant 14, e.g., the inlay component 18. For example, the implant 14 (e.g., the inlay component 18 and/or the overlay component 20) may be formed from a plastic material having a very low coefficient of friction, self-lubricating; and/or is highly resistant to abrasion such as, but not limited to, ultra-high-molecular-weight polyethylene (UHMWPE) or the like. The post 62 may include a toroidal ring 64 (best seen in FIGS. 7, 8, and 13) at a first end and a cylindrical shaft 66 having a diameter that tapers inwardly towards a second, opposite end. One or more apertures 68 may extend through a portion of the toroidal ring 64. The apertures 68 may be configured to allow the polymer material to flow into and around the toroidal ring 64 to aid in securing the post 62 to the implant 14. The cylindrical shaft 66 may have a taper inversely corresponding to the taper of a cylindrical recess formed in the anchor 16 such that a tapered connection (e.g., a Morse connection) may be formed between the post 62 and the anchor 16. It should be appreciated that while the implant 14 is shown having a tapered cylindrical post 62 and the anchor 16 includes a tapered cylindrical recess, the implant 14 may include a tapered cylindrical recess and the anchor 16 may include a tapered cylindrical post 62.

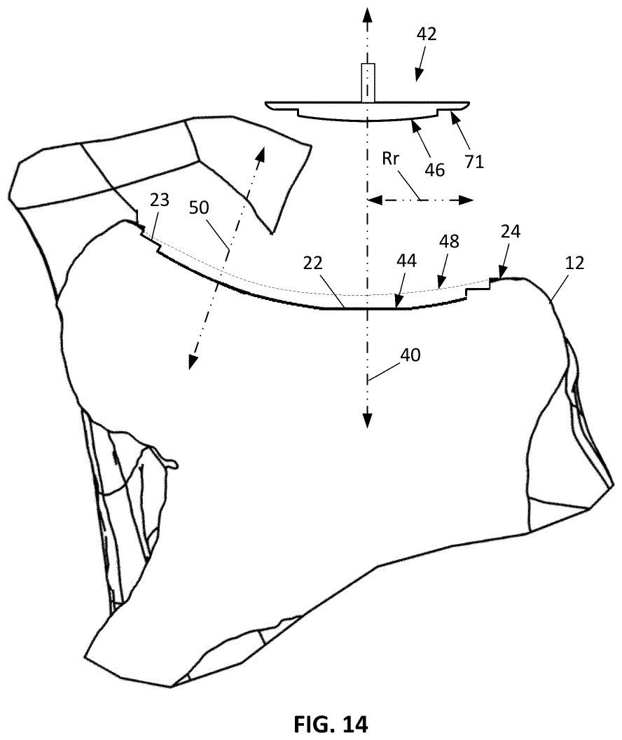

While the stepped surface 38 has been described as abutting against a portion of the patient's native articular surface 24 proximate to the excision site 22, the stepped surface 38 may alternatively (or additionally) abut against a peripheral step excision site formed proximate to the excision site 22 rather than directly against the patient's native articular surface 24. This example may be particularly useful in situations where it is difficult to form a close match between the contour/profile of the stepped surface 38 and the patient's native articular surface 24. To this end, a stepped reamer 42, FIG. 14, may be used a peripheral step excision site 23. In particular, the stepped reamer 42 may include a cutting surface 46 including a stepped region 71 proximate to the maximum radius of the cutting surface 46. The stepped region 71 may allow for a reduced and/or minimal amount of the native articular surface 24 to be removed around all or at least a portion of the periphery of the excision site 22 (e.g. primary excision site). The amount of the native articular surface 24 that is removed may be selected to provide a generally uniform mating surface for the stepped surface 38 to abut against, thereby further enhancing the resistance of the implant system 10 to dislocation from the bone 12. The stepped surface 38 may therefore have a profile/contour that substantially corresponds to the stepped region 71 of the cutting surface 46 of the reamer 42, e.g., revolved around the first axis 40.

Referring back to FIGS. 1-6, 8, and 11-12, the implant 14 may optionally include one or more retention posts 70. The retention posts 70 may be configured to be received in one or more blind holes formed in the excision site 22. The retention posts 70 may extend from one or more of the first and/or additional inlay segments 52, 56, and may aid in retaining the implant 14 to the bone 14 and generally preventing side-to-side movement of the implant 14. The retention posts 70 may optionally include one or more barbs, ribs, protrusions, or the like 72. The barbs 72 may engage the bone 12 to resist removal and/or may provide regions for bone cement to adhere to both the retention post 70 and the bone 12.

While the anchor 16 is illustrated as a separate component from the implant 14, it should be appreciated that the anchor 16 may be formed as a unitary component with the implant 14. For example, rather than the threads 72 as shown, the anchor 16 may include one or more ribs, barbs, or the like and the anchor 16 may not be removably coupled to the implant 14. The anchor 16 may be part of implant 14 such that removal of the anchor 16 cannot be accomplished without destroying the implant system 10.

The foregoing description of several methods and embodiments has been presented for purposes of illustration. It is not intended to be exhaustive or to limit the claims to the precise steps and/or forms disclosed, and obviously many modifications and variations are possible in light of the above teaching. It is intended that the scope of the invention be defined by the claims.

What is claimed is:

1. A hybrid inlay/onlay (HIO) implant system for replacing at least a portion of a patient's native articular surface on a first bone in a joint, said HIO implant system comprising:
an implant comprising:
an inlay component configured to be at least partially received in an excision site formed in said first bone beneath a removed portion of a patient's native articular surface, said inlay component comprising a bone facing surface configured to abut against said bone within said excision site; and
an onlay component comprising an uppermost surface and a stepped surface, said uppermost surface including an articulating surface configured to articulate against an articulating surface associated with a second bone of said joint and said stepped surface configured to abut against at least some of a remaining portion of said patient's native articular surface proximate to said excision site or a peripheral step excision site formed proximate to said excision site;
an anchor configured to extend away from said bone facing surface of said inlay component and to secure said HIO implant system to said first bone;
a stem extending from said bone facing surface, said stem configured to be coupled to said anchor; wherein said stem is formed from metal and wherein said implant is formed from a polymer material; and wherein said stem includes a toroidal ring at a first end and a cylindrical shaft having a diameter that tapers inwardly towards a second, opposite end, and wherein said polymer material of said implant is overmolded over said toroidal ring.

2. The HIO implant system of claim 1, wherein said onlay component includes a sidewall extending around a periphery of said onlay component between said uppermost surface and said articular surface.

3. The HIO implant system of claim 2, wherein said sidewall has a height corresponding to a distance said uppermost surface is disposed above said inlay component.

4. The HIO implant system of claim 2, wherein said sidewall has a height corresponding to a distance said uppermost surface is disposed above said remaining portion of said patient's native articular surface proximate to said excision site.

5. The HIO implant system of claim 1, wherein said onlay component has a width and a length that is greater than a width and a length of said inlay component, respectively.

6. The HIO implant system of claim 1, wherein a cross-section of said onlay component is larger than a cross-section of said inlay component.

7. The HIO implant system of claim 1, wherein said stepped surface extends along a transition between said onlay component and said inlay component.

8. The HIO implant system of claim 7, wherein said bone facing surface further comprises a plurality of undercuts.

9. The HIO implant system of claim 8, wherein said plurality of undercuts extend along a periphery of the inlay component adjacent to the articular surface.

10. The HIO implant system of claim 1, wherein said bone facing surface has a profile that generally corresponds to a profile of said excision site.

11. The HIO implant system of claim 1, wherein a first inlay segment of said bone facing surface has a profile that is revolved around a first axis.

12. The HIO implant system of claim 1, wherein said bone facing surface further comprises an additional inlay segment having a profile that is revolved around an additional axis, said additional inlay segment partially overlapping with said first inlay segment.

13. (canceled)

14. The HIO implant system of claim 1, further comprising a tapered connection between said anchor and said stem.

15. (canceled)

16. The HIO implant system of claim 1, wherein polymer material of said implant is overmolded over a portion of said stem.

17. (canceled)

18. The HIO implant system of claim 1, further comprising at least one retention post extending outward from said bone facing surface of said inlay component and configured to engage said first bone beneath said removed portion of a patient's native articular surface.

19. The HIO implant system of claim 1, wherein said inlay component and said onlay component are formed as a single piece.

20. The HIO implant system of claim 1, wherein said inlay component and said onlay component comprise two pieces coupled together.

21. The HIO implant system of claim 1, wherein said first bone comprises a glenoid, and wherein said articulating surface has a profile substantially corresponding to a profile of said patient's native articular surface which is covered by said implant.

22. The HIO implant system of claim 1, wherein said stepped surface is configured to abut against at least some of said remaining portion of said patient's native articular surface proximate to said excision site.

23. The HIO implant system of claim 1, wherein said stepped surface is configured to abut against said peripheral step excision site formed proximate to said excision site
